(19) United States
(12) Patent Application Publication
(10) Pub. No.: US 2009/0004105 A1
(43) Pub. Date: Jan. 1, 2009

(54) Molecular imaging of matrix metalloproteinase expression using labeled chlorotoxin

(76) Inventors: Zhen Cheng, Mountain View, CA (US); Sanjiv S. Gambhir, Portolla Valley, CA (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 12/215,136

(22) Filed: Jun. 25, 2008

Related U.S. Application Data
(60) Provisional application No. 60/937,315, filed on Jun. 27, 2007.

(51) Int. Cl. A61K 51/00; G01N 1/30

(52) U.S. Cl. 424/1.69; 424/1.11; 435/40.52

(57) ABSTRACT

Embodiments of the present disclosure provide for: methods for imaging MMP-2 positive tissue; methods of diagnosing the presence of one or more of MMP-2 positive precancerous cells, MMP-2 positive cancerous cells, MMP-2 positive tumor cells, and MMP-2 positive diseases in a tissue; method of monitoring the progress of one or more of MMP-2 positive precancerous cells, MMP-2 positive cancerous cells, MMP-2 positive tumor cells, and MMP-2 positive diseases in a tissue; pharmaceutical compositions for imaging one or more of MMP-2 positive precancerous cells, MMP-2 positive cancerous cells, MMP-2 positive tumor cells, and MMP-2 positive diseases; compositions; kits; and the like.

### CROSS-REFERENCE TO RELATED APPLICATION

This application claims priority to U.S. provisional application entitled, “MOLECULAR IMAGING OF MATRIX METALLOPROTEINASE EXPRESSION USING LABELED CHLOROTOXIN,” having Ser. No. 60/937,315, filed on Jun. 27, 2007, which is entirely incorporated herein by reference.

### STATEMENT REGARDING FEDERALLY SPONSORED RESEARCH OR DEVELOPMENT

This invention was made with government support under Grant No.: ICMIC P50 awarded by the National Institute of Health (NIH). The government has certain rights in the invention.

### BACKGROUND

Matrix metalloproteinases (MMPs) are zinc- and calcium-dependent enzymes that are capable of degrading the constituents of the components of the extracellular matrix such as collagens, proteoglycans, and glycoproteins. Among the different MMPs, the gelatinases (MMP-2 [gelatinase A, 72 kDa gelatinase, 72 kDa type IV collagenase] and MMP-9 [gelatinase B]) have been found to be involved in the cardiac response to ischemia and infraction, as well as in tumor invasiveness, metastasis, and angiogenesis. It also has been found that gelatinases, especially MMP-2, were over-expressed in a variety of malignant tumors, including breast, lung, brain, colon, melanoma, gastric, and esophageal carcinomas. The expression of gelatinases correlates with tumor aggressiveness and metastatic potential. Furthermore, clinical studies demonstrated that over-expression and increased activity of the MMPs result in poor prognosis in patients with different malignancies. Therefore, MMPs have become attractive targets for the development of drugs in cancer and other diseases.

PET is a diagnostic examination that involves the acquisition of physiologic images based on the detection of radiation from the emission of positrons. In particular, PET is a nuclear medicine medical imaging technique that produces a three-dimensional image or map of functional processes in the body. Positrons are tiny particles emitted from a radioactive substance administered to the patient. The subsequent images of the human body developed with this technique are used to evaluate a variety of diseases.

A short-lived radioactive tracer isotope that decays by emitting a positron is chemically incorporated into a molecule (e.g., a biologically active molecule, a polypeptide, or polynucleotide) and is injected into the living subject (e.g., usually into blood circulation). There is a waiting period while the molecule becomes concentrated in tissues of interest, then the subject is placed in the imaging scanner. The short-lived isotope decays, emitting a positron. After travelling up to a few millimeters, the positron annihilates with an electron, producing a pair of annihilation photons (similar to gamma rays) moving in opposite directions. These are detected when they reach a scintillator material in the scanning device, creating a burst of light that is detected by photomultiplier tubes. The technique depends on simultaneous or coincident detection of the pair of photons: photons that do not arrive in pairs (e.g., within a few nanoseconds) are ignored.

Because annihilation photons are always emitted 180° apart, it is possible to localize their source to a straight-line in space. Using statistics collected from tens-of-thousands of coincidence events, a map of their origin in the body can be plotted. The resulting map shows the tissues in which the molecular probe has become concentrated and can be interpreted by nuclear medicine physician or radiologist in the context of the patient's diagnosis and treatment plan. PET is used heavily in clinical oncology (medical imaging of tumors and the search for metastases) and in human brain and heart research.

Because PET imaging is most useful in combination with anatomical imaging, such as CT, modern PET scanners are now available with integrated high-end multi-detector-row CT scanners. Because the two scans can be performed simultaneously, not only is time saved, but also the two sets of images are precisely registered so that areas of abnormality on the PET imaging can be correlated with anatomy on the CT images.

Another imaging system includes single photon emission computed tomography (SPECT). SPECT is a nuclear medicine tomographic imaging technique using gamma rays. It is very similar to conventional nuclear medicine planar imaging using a gamma camera. However, it is able to provide true 3-dimensional information. This information is typically presented as cross-sectional slices through the patient, but can be freely reformatted or manipulated as required. In the similar way as a plain X-ray is a 2-dimensional view of a 3-dimensional structure, the image obtained by a gamma camera image is a 2-dimensional view of 3-dimensional distribution of a radionuclide.

SPECT imaging is performed by using a gamma camera to acquire multiple images (also called projections) from multiple angles. A computer can then be used to apply a tomographic reconstruction algorithm to the multiple projections, yielding a 3D dataset. To acquire SPECT images, the gamma camera(s) is rotated around the patient. Projections are acquired at defined points during the rotation, typically every 3-6°. In most cases, a full 360° rotation is used to obtain an optimal reconstruction.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

The patent or application file contains at least one drawing executed in color. Copies of this patent or patent application publication with color drawing(s) will be provided by the Office upon request and payment of the necessary fee.

FIG. 1 illustrates the amino acid sequence of chlorotoxin (three-letter code and one-letter code).

FIG. 2 illustrates an embodiment of a synthetic scheme for forming 4-[¹⁸F]Fluorobenzoate chlorotoxin.

FIG. 3 illustrates the biodistribution data (FIG. 3A) and tumor to normal organ ratio (FIG. 3B) for ¹⁸F-FB-Cltx in nude mice bearing subcutaneously xenotransplanted MBA-MB-435. Biodistribution data are expressed as normalized accumulation of activity in % ID/g±SD (n=3).

FIG. 4 illustrates the biodistribution data (FIG. 4A) and tumor to normal organ ratio (FIG. 4B) for ¹⁸F-FB-Cltx in nude mice bearing subcutaneously xenotransplanted B16F10. Biodistribution data are expressed as normalized accumulation of activity in % ID/g±SD (n=3).

FIG. 5 illustrates digital images that show the decay-corrected whole-body coronal and transverse microPET images of normal nude mice bearing U87MG (FIG. 5A), C6 (FIG. 5B), MDA-MB-435 (FIG. 5C) or B16F10 (FIG. 5D) at 0.5, 1, 2, and 3 hr (10-min static image) after injection of 50 μCi of ¹⁸F-FB-Cltx. Arrows indicated location of tumors.

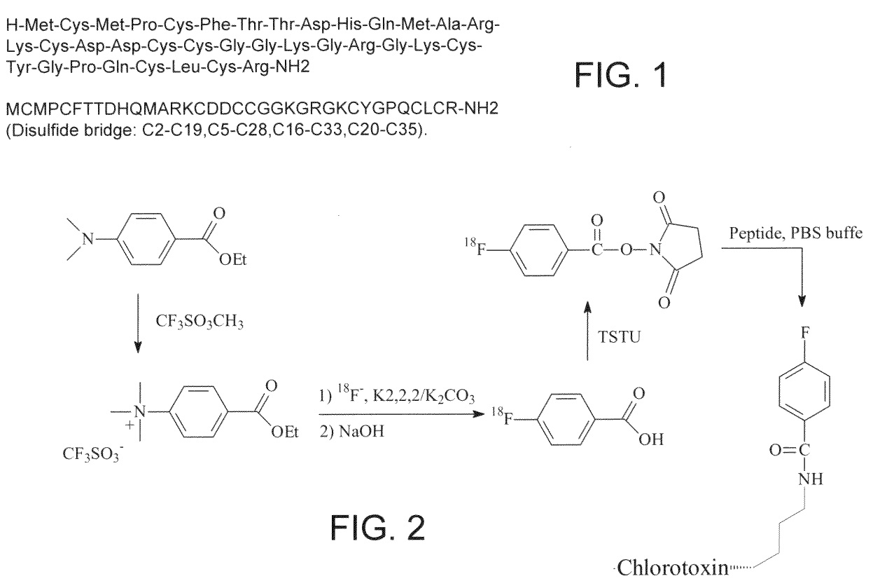

### SUMMARY

Embodiments of the present disclosure provide for: methods for imaging MMP-2 positive tissue; methods of diagnosing the presence of one or more of MMP-2 positive precancerous cells, MMP-2 positive cancerous cells, MMP-2 positive tumor cells, and MMP-2 positive diseases in a tissue; methods of monitoring the progress of one or more of MMP-2 positive precancerous cells, MMP-2 positive cancerous cells, MMP-2 positive tumor cells, and MMP-2 positive diseases in a tissue; pharmaceutical compositions for imaging one or more of MMP-2 positive precancerous cells, MMP-2 positive cancerous cells, MMP-2 positive tumor cells, and MMP-2 positive diseases; compositions; kits; and the like.

Embodiments of the method for imaging MMP-2 positive tissue, among others, includes: contacting a MMP-2 positive tissue with a labeled chlorotoxin; and imaging the tissue with an imaging system.

Embodiments of the method of diagnosing the presence of one or more of MMP-2 positive precancerous cells, MMP-2 positive cancerous cells, MMP-2 positive tumor cells, and MMP-2 positive diseases in a tissue, among others, includes: contacting a tissue with a labeled chlorotoxin; and imaging the tissue with an imaging system.

Embodiments of the method of diagnosing the presence of one or more of MMP-2 positive precancerous cells, MMP-2 positive cancerous cells, MMP-2 positive tumor cells, and MMP-2 positive diseases in a tissue, among others, includes: contacting a MMP-2 positive tissue with a labeled chlorotoxin; and imaging the MMP-2 positive tissue with an imaging system.

Embodiments of the pharmaceutical composition, among others, includes: a labeled chlorotoxin.

Embodiments of the composition, among others, includes: a labeled chlorotoxin.

Embodiments of the kit, among others, includes: a labeled chlorotoxin and directions for use.

In an embodiment, the labeled chlorotoxin is a radio-labeled chlorotoxin. The radio-label is selected from: ¹⁸F, ¹²⁴I, ¹²³I, ¹²⁵I, ¹³¹I, ^{76/77}Br, ⁶⁴Cu, ⁸⁶Y, ⁸⁹Zr, ⁶⁸Ga, ⁹⁹Tc, ¹¹¹In, ^{186/188}Re, ¹⁷⁷Lu, ¹⁵³Sm, or ⁹⁰Y. In an embodiment, the labeled chlorotoxin is a ¹⁸F-labeled chlorotoxin, analogs thereof, portions thereof, mutants thereof, or varients thereof. In an embodiment, the labeled chlorotoxin is a ¹⁸F-labeled chlorotoxin. In an embodiment, the labeled chlorotoxin is labeled with ¹⁸F by coupling the chlorotoxin with N-succinimidyl-4-¹⁸F-fluorobenzoate (¹⁸F-SFB), wherein chlorotoxin has an amino acid sequence SEQ ID NO: 1

These embodiments, uses of these embodiments, and other uses, features and advantages of the present disclosure, will become more apparent to those of ordinary skill in the relevant art when the following detailed description of the preferred embodiments is read in conjunction with the appended figures.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of synthetic organic chemistry, biochemistry, biology, molecular biology, recombinant DNA techniques, pharmacology, imaging, and the like, which are within the skill of the art. Such techniques are explained fully in the literature. In particular, See, e.g., Maniatis, Fritsch & Sambrook, “Molecular Cloning: A Laboratory Manual (1982); “DNA Cloning: A Practical Approach,” Volumes I and II (D. N. Glover ed. 1985); “Oligonucleotide Synthesis” (M. J. Gait ed. 1984); “Nucleic Acid Hybridization” (B. D. Hames & S. J. Higgins eds. (1985)); “Transcription and Translation” (B. D. Hames & S. J. Higgins eds. (1984)); “Animal Cell Culture” (R. I. Freshney, ed. (1986)); “Immobilized Cells and Enzymes” (IRL Press, (1986)); B. Perbal, “A Practical Guide To Molecular Cloning” (1984), each of which is incorporated herein by reference.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a compound” includes a plurality of compounds. In this specification and in the claims that follow, reference will be made to a number of terms that shall be defined to have the following meanings unless a contrary intention is apparent.

### DEFINITIONS

In describing and claiming the disclosed subject matter, the following terminology will be used in accordance with the definitions set forth below.

Chlorotoxin (Cltx) is a 36-amino acid peptide (SEQ ID NO: 1) that was originally isolated from scorpion venom (DeBin, J. A., and Strichartz, G. R. Chloride channel inhibition by the venom of the scorpion Leiurus quinquestriatus. Toxicon. 1991; 29, 1403-1408, which is incorporated herein by reference). Immunohistochemical studies show that Cltx specifically and selectively binds to tumors of neuroectodermal origin (Lyons SA, O'Neal J, Sontheimer H. Chlorotoxin, a scorpion-derived peptide, specifically binds to gliomas and tumors of neuroectodermal origin. Glia. 2002; 39: 162-173, which is incorporated herein by reference). Further studies demonstrated that Cltx is a specific MMP-2 inhibitor and can bind with MMP-2 presented on the surface of glioma cells in high affinity (Deshane J, Garner C C, Sontheimer H. Chlorotoxin inhibits glioma cell invasion via matrix metalloproteinase-2. J Biol Chem. 2003; 278: 4135-4144, which is incorporated herein by reference). A synthetic version of Cltx, TM-601, has been shown to selectively localize in human gliomas in vivo.

Fluorine-18 (t_{1/2}=109.7 min; β⁺, 99%) is an ideal short-lived PET isotope for labeling small molecular recognition units such as the antigen binding domain of antibody fragments and small biologically active peptides. ¹⁸F-labeled prosthetic groups such as N-succinimidyl 4-¹⁸F-fluorobenzoate (¹⁸F-SFB) have been developed that can be attached to either N-terminal or lysine ε-amino groups with little or no loss of bioactivity of the peptide ligand (e.g., Cltx).

The term “polymer” means any compound that is made up of two or more monomeric units covalently bonded to each other, where the monomeric units may be the same or different, such that the polymer may be a homopolymer or a heteropolymer. Representative polymers include peptides, polysaccharides, nucleic acids and the like, where the polymers may be naturally occurring or synthetic.

The term “polypeptides” includes proteins and fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

“Variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of this disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly, where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

“Identity,” as known in the art, is a relationship between two or more polypeptide sequences, as determined by comparing the sequences. In the art, “identity” also refers to the degree of sequence relatedness between polypeptides as determined by the match between strings of such sequences. “Identity” and “similarity” can be readily calculated by known methods, including, but not limited to, those described in Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073, (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present disclosure.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence, or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

Conservative amino acid variants can also comprise non-canonical amino acid residues. Non-canonical amino acids include, without limitation, trans-3-methylproline, 2,4-methanoproline, cis-4-hydroxyproline, trans-4-hydroxyproline, N-methyl-glycine, allo-threonine, methylthreonine, hydroxy-ethylcysteine, hydroxyethylhomocysteine, nitro-glutamine, homoglutamine, pipecolic acid, thiazolidine carboxylic acid, dehydroproline, 3- and 4-methylproline, 3,3-dimethylproline, tert-leucine, norvaline, 2-azaphenyl-alanine, 3-azaphenylalanine, 4-azaphenylalanine, and 4-fluorophenylalanine. Several methods are known in the art for incorporating non-canonical amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Proteins are purified by chromatography. (Robertson, et al., J. Am. Chem. Soc., 113: 2722, 1991; Ellman, et al., Methods Enzymol., 202: 301, 1991; Chung, et al., Science, 259: 806-9, 1993; and Chung, et al., Proc. Natl. Acad. Sci. USA, 90: 10145-9, 1993). In a second method, translation is carried out in Xenopus oocytes by microinjection of mutated mRNA and chemically aminoacylated suppressor tRNAs (Turcatti, et al., J. Biol. Chem., 271: 19991-8, 1996). Within a third method, E. coli cells are cultured in the absence of a natural amino acid that is to be replaced (e.g., phenylalanine) and in the presence of the desired non-canonical amino acid(s) (e.g., 2-azaphenylalanine, 3-azaphenylalanine, 4-azaphenylalanine, or 4-fluorophenylalanine). The non-canonical amino acid is incorporated into the protein in place of its natural counterpart. (Koide, et al., Biochem., 33: 7470-6, 1994). Naturally occurring amino acid residues can be converted to non-canonical species by in vitro chemical modification. Chemical modification can be combined with site-directed mutagenesis to further expand the range of substitutions (Wynn, et al., Protein Sci., 2: 395-403, 1993).

A “fragment” of a molecule such as a protein or nucleic acid is meant to refer to any portion of the amino acid or nucleotide genetic sequence.

“DNA” refers to the polymeric form of deoxyribonucleotides (adenine, guanine, thymine, or cytosine) in either single stranded form, or as a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but advantageously is double-stranded DNA. An “isolated” nucleic acid molecule is one that is separated from other nucleic acid molecules that are present in the natural source of the nucleic acid. A “nucleoside” refers to a base linked to a sugar. The base may be adenine (A), guanine (G) (or its substitute, inosine (I)), cytosine (C), or thymine (T) (or its substitute, uracil (U)). The sugar may be ribose (the sugar of a natural nucleotide in RNA) or 2-deoxyribose (the sugar of a natural nucleotide in DNA). A “nucleotide” refers to a nucleoside linked to a single phosphate group.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides may be chemically synthesized and may be used as primers or probes. Oligonucleotide means any nucleotide of more than 3 bases in length used to facilitate detection or identification of a target nucleic acid, including probes and primers.

As used herein, the term “polynucleotide” generally refers to any polyribonucleotide or polydeoxyribonucleotide, which may be unmodified RNA or DNA or modified RNA or DNA. Thus, for instance, polynucleotides as used herein refers to, among others, single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically, double-stranded or a mixture of single- and double-stranded regions. Polynucleotide encompasses the terms “nucleic acid,” “nucleic acid sequence,” or “oligonucleotide” as defined above.

As used herein, the term polynucleotide includes DNAs or RNAs as described above that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein.

It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including simple and complex cells, inter alias.

By way of example, a polynucleotide sequence of the present disclosure may be identical to the reference sequence, that is be 100% identical, or it may include up to a certain integer number of nucleotide alterations as compared to the reference sequence. Such alterations are selected from the group including at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence. The number of nucleotide alterations is determined by multiplying the total number of nucleotides in the reference nucleotide by the numerical percent of the respective percent identity (divided by 100) and subtracting that product from said total number of nucleotides in the reference nucleotide. Alterations of a polynucleotide sequence encoding the polypeptide may alter the polypeptide encoded by the polynucleotide following such alterations.

The term “codon” means a specific triplet of mononucleotides in the DNA chain. Codons correspond to specific amino acids (as defined by the transfer RNAs) or to the start and stop of translation by the ribosome.

The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (e.g., GAU and GAC triplets each encode Asp).

The DNA encoding the protein disclosed herein can be prepared by the usual chemical synthesis methods as are known in the art. An “expression vector” is useful for expressing the DNA encoding the protein used herein and for producing the protein. The expression vector is not limited as long as it expresses the gene encoding the protein in various prokaryotic and/or eukaryotic host cells and produces this protein. Examples thereof are pMAL C2, pEF-BOS (Nucleic Acids Res. 18:5322 (1990) and so on), pME18S (Experimental Medicine: SUPPLEMENT, “Handbook of Genetic Engineering” (1992)), etc.

When bacteria, particularly E. coli are used as host cells, an expression vector generally comprises, at least, a promoter/operator region, an initiation codon, the DNA encoding the protein termination codon, terminator region, and replicon.

When yeast, animal cells, or insect cells are used as hosts, an expression vector includes, at least, a promoter, an initiation codon, the DNA encoding the protein and a termination codon. It may also comprise the DNA encoding a signal peptide, enhancer sequence, 5′- and 3′-untranslated region of the gene encoding the protein, splicing junctions, polyadenylation site, selectable marker region, and replicon. The expression vector may also contain, if required, a gene for gene amplification (marker) that is usually used.

A promoter/operator region to express the protein in bacteria comprises a promoter, an operator, and a Shine-Dalgarno (SD) sequence (e.g., AAGG). For example, when the host is Escherichia, it preferably comprises Trp promoter, lac promoter, recA promoter, lambda PL promoter, b 1pp promoter, tac promoter, or the like. Examples of a promoter to express the protein in yeast are PH05 promoter, PGK promoter, GAP promoter, ADH promoter, and so on. When the host is Bacillus, examples thereof are SL01 promoter, SP02 promoter, penP promoter, and so on. When the host is a eukaryotic cell such as a mammalian cell, examples thereof are SV40-derived promoter, a retrovirus promoter, heat shock promoter, and so on, and preferably SV-40 and retrovirus-derived one. As a matter of course, the promoter is not limited to the above examples. In addition, using an enhancer is effective for expression.

A preferable initiation codon is, for example, a methionine codon (ATG).

A commonly used termination codon (e.g., TAG, TAA, TGA) is exemplified as a termination codon. Usually, natural or synthetic terminators are used as a terminator region.

A “replicon” means a DNA capable of replicating the whole DNA sequence in host cells, and includes a natural plasmid, an artificially modified plasmid (DNA fragment prepared from a natural plasmid), a synthetic plasmid, and so on. Examples of preferable plasmids are pBR322 or its artificial derivatives (DNA fragment obtained by treating pBR322 with appropriate restriction enzymes) for E. coli, yeast plasmid or yeast chromosomal DNA for yeast, and pRSVneo ATCC 37198, pSV2dhfr ATCC 37145, pdBPV-MMTneo ATCC 37224, pSV2neo ATCC 37149, and such for mammalian cells.

An enhancer sequence, polyadenylation site, and splicing junction that are usually used in the art, such as those derived from SV40 can also be used.

A selectable marker usually employed can be used according to the usual method. Examples thereof are resistance genes for antibiotics, such as tetracycline, ampicillin, or kanamycin.

Examples of genes for gene amplification are dihydrofolate reductase (DHFR) gene, thymidine kinase gene, neomycin resistance gene, glutamate synthase gene, adenosine deaminase gene, ornithine decarboxylase gene, hygromycin-B-phophotransferase gene, aspartate transcarbamylase gene, etc. It should also be noted that these are also selection genes, except for use in mammalian cells instead of the genes described in the paragraph above, which are used in bacteria. Usually the genes described in the paragraph above are used for plasmid amplification in bacterial cells, and the ones in this paragraph are used for selection of mammalian cells.

The expression vector used herein can be prepared by continuously and circularly linking at least the above-mentioned promoter, initiation codon, DNA encoding the protein, termination codon, and terminator region, to an appropriate replicon. If desired, appropriate DNA fragments (for example, linkers, restriction sites, and so on), can be used by the usual method such as digestion with a restriction enzyme and ligation using T4 DNA ligase.

As used herein, “transformants” can be prepared by introducing the expression vector mentioned above into host cells.

As used herein, “host” cells are not limited as long as they are compatible with an expression vector mentioned above and can be transformed. Examples thereof are various cells such as wild-type cells or artificially established recombinant cells usually used in the technical field (e.g., bacteria (Escherichia and Bacillus), yeast (e.g., Saccharomyces, Pichia, and such), animal cells, or insect cells).

Specific examples are E. coli (e.g., DH5ₐₗₚₕₐ, TB1, HB101, and such), mouse-derived cells (e.g., COP, L, C127, Sp2/0, NS-1, NIH 3T3, and such), rat-derived cells (e.g., PC12, PC12h), hamster-derived cells (e.g., BHK, CHO, and such), monkey-derived cells (e.g., COS1, COS3, COS7, CV1, Velo, and such), and human-derived cells (Hela, diploid fibroblast-derived cells, myeloma cells, and HepG2, and such).

An expression vector can be introduced (transformed/transfected/transduced/electroporated) into host cells by known methods.

Transformation can be performed, for example, according to the method of Cohen et al. (Proc. Natl. Acad. Sci. USA, 69:2110 (1972)), protoplast method (Mol, Gen. Genet., 168:111 (1979)), or competent method (J. Mol. Biol., 56:209 (1971)) when the hosts are bacteria (E. coli, Bacillus subtilis, and such), the method of Hinnen et al. (Proc. Natl. Acad. Sci. USA, 75:1927 (1978)), or lithium method (J. Bacteriol., 153:163 (1983)) when the host is Saccharomyces cerevisiae, the method of Graham (Virology, 52:456 (1973)) when the hosts are animal cells, and the method of Summers et al. (Mol. Cell. Biol., 3:2156-2165 (1983)) when the hosts are insect cells.

The protein disclosed herein, can be produced by cultivating transformants (in the following, this term includes transfectants) comprising an expression vector prepared as mentioned in nutrient media.

The nutrient media preferably comprise carbon source, inorganic nitrogen source, or organic nitrogen source necessary for the growth of host cells (transformants). Examples of the carbon source are glucose, dextran, soluble starch, and sucrose, and examples of the inorganic or organic nitrogen source are ammonium salts, nitrates, amino acids, corn steep liquor, peptone, casein, meet extract, soy bean cake, and potato extract. If desired, they may comprise other nutrients (for example, an inorganic salt (for example, calcium chloride, sodium dihydrogenphosphate, and magnesium chloride), vitamins, antibiotics (for example, tetracycline, neomycin, ampicillin, kanamycin, and so on).

Cultivation of cell lines is performed by a method known in the art. Cultivation conditions such as temperature, pH of the media, and cultivation time are selected appropriately so that the protein is produced in large quantities.

Examples of the isolation and purification method are a method utilizing solubility, such as salting out and solvent precipitation method; a method utilizing the difference in molecular weight, such as dialysis, ultrafiltration, gel filtration, and sodium dodecyl sulfate-polyacrylamide gel electrophoresis; a method utilizing charges, such as ion exchange chromatography and hydroxylapatite chromatography; a method utilizing specific affinity, such as affinity column chromatography; a method utilizing the difference in hydrophobicity, such as reverse phase high performance liquid chromatography; and a method utilizing the difference in isoelectric point, such as isoelectric focusing.

As used herein the term “isolated” is meant to describe a polynucleotide, a polypeptide, an antibody, or a host cell that is in an environment different from that in which the polynucleotide, the polypeptide, the antibody, or the host cell naturally occurs.

“Optional” or “optionally” means that the subsequently described circumstance may or may not occur, so that the description includes instances where the circumstance occurs and instances where it does not.

“Hybridizing” and “binding”, with respect to polynucleotides, are used interchangeably. The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions.

The term “salts” herein refers to both salts of carboxyl groups and to acid addition salts of amino groups of the polypeptides of the present disclosure. Salts of a carboxyl group may be formed by methods known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids such as, for example, hydrochloric acid or sulfuric acid, and salts with organic acids such as, for example, acetic acid or oxalic acid. Any of such salts should have substantially similar activity to the peptides and polypeptides of the present disclosure or their analogs.

By “administration” is meant introducing a compound of the present disclosure into a subject. Any route of administration, such as oral, topical, subcutaneous, peritoneal, intraarterial, inhalation, vaginal, rectal, nasal, introduction into the cerebrospinal fluid, or instillation into body compartments, could be used.

As used herein, the term “host” or “organism” includes humans, mammals (e.g., cats, dogs, horses, etc.), living cells, and other living organisms. A living organism can be as simple as, for example, a single eukaryotic cell or as complex as a mammal. Typical hosts to which embodiments of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. Additionally, for in vitro applications, such as in vitro diagnostic and research applications, body fluids and cell samples of the above subjects will be suitable for use, such as mammalian (particularly primate such as human) blood, urine, or tissue samples, or blood, urine, or tissue samples of the animals mentioned for veterinary applications. In some embodiments, a system includes a sample and a host. The term “living host” refers to host or organisms noted above that are alive and are not dead. The term “living host” refers to the entire host or organism and not just a part excised (e.g., a liver or other organ) from the living host.

The term “detectable signal” is a signal derived from non-invasive imaging techniques such as, but not limited to, positron emission tomography (PET), single photon emission computed tomography (SPECT), and/or magnetic resonance imaging (MRI). The detectable signal is detectable and distinguishable from other background signals that may be generated from the host. In other words, there is a measurable and statistically significant difference (e.g., a statistically significant difference is enough of a difference to distinguish among the detectable signal and the background, such as about 0.1%, 1%, 3%, 5%, 10%, 15%, 20%, 25%, 30%, or 40% or more difference between the detectable signal and the background) between detectable signal and the background. Standards and/or calibration curves can be used to determine the relative intensity of the detectable signal and/or the background.

The signal can be generated from one or more compounds of the present disclosure. In an embodiment, the signal may need to be sum of each of the individual compounds. In an embodiment, the signal can be generated from a summation, an integration, or other mathematical process, formula, or algorithm, where the signal is from one or more compounds. In an embodiment, the summation, the integration, or other mathematical process, formula, or algorithm can be used to generate the signal so that the signal can be distinguished from background noise and the like.

The detectable signal is defined as an amount sufficient to yield an acceptable image using equipment that is available for pre-clinical use. A detectable signal maybe generated by one or more administrations of the compounds of the present disclosure. The amount of the administered compound of the present disclosure can vary according to factors such as the degree of susceptibility of the individual, the age, sex, and weight of the individual, idiosyncratic responses of the individual, the dosimetry, and the like. The amount of the compounds of the present disclosure can also vary according to instrument and digital processing related factors.

### General Discussion

Labeled (e.g., radiolabeled) chlorotoxin and analogs thereof; pharmaceutical compositions including radiolabeled chlorotoxin and analogs thereof; methods of making radiolabeled chlorotoxin; methods for imaging more matrix metalloproteinase (MMP-2) positive tissue in vivo and/or in vitro; methods for imaging MMP-2 positive diseases in vivo and/or in vitro; methods of monitoring the progress of one or more MMP-2 positive precancerous cells, MMP-2 positive cancerous cells, and MMP-2 positive tumor cells in vivo and/or in vitro; pharmaceutical compositions for imaging MMP-2 positive precancerous cells, MMP-2 positive cancerous cells, and MMP-2 positive tumor cells; kits including radiolabeled chlorotoxin; and the like are disclosed. It should be noted that “MMP-2 positive” means that MMP-2 is over expressed and is correlated to certain disease states.

In addition, the present disclosure includes compositions used in and methods relating to non-invasive imaging (e.g., positron emission topography (PET) and/or single photon emission computed tomography (SPECT)) of matrix MMP-2 positive tissue, cancer, and tumors in vivo. In particular, the composition and/or pharmaceutical composition include a ¹⁸F-labeled chlorotoxin (SEQ ID NO: 1) that is used in PET imaging of MMP-2 positive tissue, cancer, and tumors in vivo. Embodiments of the present disclosure can be used to detect a detectable signal corresponding to the labeled chlorotoxin. The detectable signal can be used to produce an image corresponding to the matrix MMP-2 positive tissue, cancer, and tumors.

The present disclosure includes methods relating to in vivo and/or in vitro non-invasive PET or SPECT radiolabeled chlorotoxin. The ability to noninvasively and quantitatively detect and image using radiolabeled chlorotoxin can assist in early and sensitive cancer detection and patient selection for clinical trials based on in vivo expression quantification as well as allow early tumor diagnosis and patient stratification, better treatment monitoring, dose optimization, and the like.

In an embodiment, the isotope in the radiolabeled chlorotoxin is a PET isotope (e.g., ¹⁸F). Although currently y emitters are more readily available and have longer half-lives relative to positron emitting radionucleotides, PET cameras allow electronic rather than mechanical collimation of incoming photons by recording the coincidence of simultaneous pairs of annihilation photons (511 keV per photon) at opposite detectors.

Imaging gelatinase activity and expression using a noninvasive methodology (PET or SPECT), may provide unique techniques 1) to diagnosis diseases that over-express MMPs (e.g., MMP-2); 2) to predict the metastatic potential of a MMP-2 tumor; 3) to monitor the therapeutic efficacy of MMP-2 inhibitors and other drugs; and/or 4) to help for the optimization of the dosage for an efficient MMP-2 targeted treatment.

“Cancer”, “tumor”, and “precancerous” as used herein, shall be given their ordinary meaning, as general terms for diseases in which abnormal cells divide without control. Cancer cells can invade nearby tissues and can spread through the bloodstream and lymphatic system to other parts of the body. In an embodiment, “cancer”, “tumor”, and “precancerous” refer to MMP-2 positive cancer, tumors, precancerous tissues, and the like. In an embodiment, the MMP-2 positive cancer, tumors, precancerous tissues, and the like, correspond to malignant tumors, including breast, lung, brain, colon, melanoma, gastric, and esophageal carcinomas.

There are several main types of cancer, for example, carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma is cancer that begins in the cells of the immune system.

When normal cells lose their ability to behave as a specified, controlled and coordinated unit, a tumor is formed. Generally, a solid tumor is an abnormal mass of tissue that usually does not contain cysts or liquid areas (some brain tumors do have cysts and central necrotic areas filled with liquid). A single tumor may even have different populations of cells within it, with differing processes that have gone awry. Solid tumors may be benign (not cancerous), or malignant (cancerous). Different types of solid tumors are named for the type of cells that form them. Examples of solid tumors are sarcomas, carcinomas, and lymphomas. Leukemias (cancers of the blood) generally do not form solid tumors.

Representative cancers include, but are not limited to, cancer of the head, neck, eye, mouth, throat, esophagus, chest, bone, lung, colon, rectum, stomach, prostate, breast, ovaries, kidney, liver, pancreas, and brain. Additional cancers include, but are not limited to, the following: leukemias such as, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as, but not limited to, chronic myelocytic (granulocytic) leukemia, chronic lymphocytic leukemia, hairy cell leukemia; polycythemia vera; lymphomas such as, but not limited to, Hodgkin's disease, non-Hodgkin's disease; multiple myelomas such as, but not limited to, smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone cancer and connective tissue sarcomas such as, but not limited to, bone sarcoma, myeloma bone disease, multiple myeloma, cholesteatoma-induced bone osteosarcoma, Paget's disease of bone, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, and synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma; breast cancer including, but not limited to, adenocarcinoma, lobular (small cell) carcinoma, intraductal carcinoma, medullary breast cancer, mucinous breast cancer, tubular breast cancer, papillary breast cancer, Paget's disease (including juvenile Paget's disease) and inflammatory breast cancer; adrenal cancer such as, but not limited to, pheochromocytoma and adrenocortical carcinoma; thyroid cancer such as, but not limited to, papillary or follicular thyroid cancer, medullary thyroid cancer and anaplastic thyroid cancer; pancreatic cancer such as, but not limited to, insulinoma, gastrinoma, glucagonoma, vipoma, somatostatin-secreting tumor, and carcinoid or islet cell tumor; pituitary cancers such as, but not limited to, Cushing's disease, prolactin-secreting tumor, acromegaly, and diabetes insipidus; eye cancers such as, but not limited to, ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as, but not limited to, squamous cell carcinoma, and adenocarcinoma; uterine cancers such as, but not limited to, endometrial carcinoma and uterine sarcoma; ovarian cancers such as, but not limited to, ovarian epithelial carcinoma, borderline tumor, germ cell tumor, and stromal tumor; esophageal cancers such as, but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as, but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as, but not limited to, hepatocellular carcinoma and hepatoblastoma, gallbladder cancers such as adenocarcinoma; cholangiocarcinomas such as, but not limited to, papillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as, but not limited to, germinal tumor, seminoma, anaplastic, classic (typical), spermatocytic, nonseminoma, embryonal carcinoma, teratoma carcinoma, choriocarcinoma (yolk-sac tumor), prostate cancers such as, but not limited to, adenocarcinoma, leiomyosarcoma, and rhabdomyosarcoma; penal cancers; oral cancers such as, but not limited to, squamous cell carcinoma; basal cancers; salivary gland cancers such as, but not limited to, adenocarcinoma, mucoepidermoid carcinoma, and adenoidcystic carcinoma; pharynx cancers such as, but not limited to, squamous cell cancer, and verrucous; skin cancers such as, but not limited to, basal cell carcinoma, squamous cell carcinoma and melanoma, superficial spreading melanoma, nodular melanoma, lentigo malignant melanoma, acral lentiginous melanoma; kidney cancers such as, but not limited to, renal cell cancer, adenocarcinoma, hypernephroma, fibrosarcoma, transitional cell cancer (renal pelvis and/or ureter); Wilms' tumor; bladder cancers such as, but not limited to, transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., inc., United States of America). It is also contemplated that cancers caused by aberrations in apoptosis can also be treated by the methods and compositions of the present disclosure. Such cancers may include, but not be limited to, follicular lymphomas, carcinomas with p53 mutations, hormone dependent tumors of the breast, prostate and ovary, and precancerous lesions such as familial adenomatous polyposis, and myelodysplastic syndromes.

A tumor can be classified as malignant or benign. In both cases, there is an abnormal aggregation and proliferation of cells. In the case of a malignant tumor, these cells behave more aggressively, acquiring properties of increased invasiveness. Ultimately, the tumor cells may even gain the ability to break away from the microscopic environment in which they originated, spread to another area of the body (with a very different environment, not normally conducive to their growth), and continue their rapid growth and division in this new location. This is called metastasis. Once malignant cells have metastasized, achieving a cure is more difficult.

Benign tumors have less of a tendency to invade and are less likely to metastasize. Brain tumors spread extensively within the brain but do not usually metastasize outside the brain. Gliomas are very invasive inside the brain, even crossing hemispheres. They do divide in an uncontrolled manner, though. Depending on their location, they can be just as life threatening as malignant lesions. An example of this would be a benign tumor in the brain, which can grow and occupy space within the skull, leading to increased pressure on the brain.

It should be noted that cancerous cells, cancer, and tumors are sometimes used interchangeably in the disclosure.

In addition, embodiments of the present disclosure can be used to image, monitor, and diagnose MMP-2 positive diseases. MMP-2 positive diseases include, but are not limited to, tumor, myocardial infarction, rheumatoid arthritis, and atherosclerosis.

### Labeled Chlorotoxin

In general, embodiments of the present disclosure include labeled chlorotoxin. The label can include, but is not limited to, ¹⁸F, ¹²⁴I, ¹²³I, ¹²⁵I, ¹³¹I, ^{76/77}Br, ⁶⁴Cu, ⁸⁶Y, ⁸⁹Zr, ⁶⁸Ga, ⁹⁹Tc, ¹¹¹In, ^{186/188}Re, ¹⁷⁷Lu, ¹⁵³Sm, and ⁹⁰Y. In addition, the label can include, but is not limited to, fluorescent moieties (e.g., fluorescein, methylene blue, PHOTOFRIN®, Lutrin, ANTRIN®, FOSCAN®, aminolevulinic acid, aluminum (III) phthalocyanine tetrasulfonate, Hypericin, verteporfin, and the like) and cytotoxic moieties (e.g., gelonin, ricin, saponin, pseudonomas exotoxin, pokeweed antiviral protein, diphtheria toxin, and the like). In an embodiment, the label is ¹⁸F. The labels can be attached directly or indirectly to the chlorotoxin. In an embodiment, the labels can be attached to chlorotoxin via a chemical compound that bonds with the chlorotoxin. In another embodiment, the labels can be attached using a chelator (e.g., a macrocyclic chelator, a non-cyclic chelator, and an amino acid chelator).

In another embodiment, the label is a contrast agent for imaging using MRI. As used herein, a “contrast agent” is intended to include any agent that is physiologically tolerable and capable of providing enhanced contrast for magnetic resonance imaging. Contrast agents typically have the capability of altering the response of a tissue to magnetic fields. Contrast agents include paramagnetic agents, e.g., a gadolinium-chelating group complex, such as gadolinium-diethylenetriamine penta-acetic acid, or a manganese chelating group complex; or biologically compatible superparamagnetic agents such as iron oxide. Contrast agents, such as those described in U.S. Pat. No. 4,687,658; U.S. Pat. No. 5,314,680; and U.S. Pat. No. 4,976,950 can be used in preparing the compositions of the present disclosure, and are included herein by reference. Contrast agents are commercially available (e.g., the gadolinium chelate Prohance™ is available from Squibb, and the gadolinium chelate Dotarem™ is available from Guerbet).

In particular, embodiments of the present disclosure include a ¹⁸F-labeled chlorotoxin, analogs thereof, portions thereof, mutants thereof, and varients thereof. ¹⁸F has a high positron efficiency and short positron-range and is a low radiation dose for patients. The chlorotoxin can include, but is not limited to, the peptide of SEQ ID NO: 1, analogs thereof, portions thereof, mutants thereof, and varients thereof.

In an embodiment, the present disclosure includes, but is not limited to, a ¹⁸F-labeled chlorotoxin (SEQ ID NO: 1) (also referred to as ¹⁸F-FB-Cltx). Additional details regarding the ¹⁸F-labeled chlorotoxin are described in Example 1.

The term “mutant” is employed broadly to refer to a protein that differs in some way from a reference wild-type protein, where the protein may retain biological properties of the reference wild-type (e.g., naturally occurring) protein, or may have biological properties that differ from the reference wild-type protein. The term “biological property” of the subject proteins includes, but is not limited to, interaction with MMP-2, in vivo and/or in vitro stability (e.g., half-life), and the like. Mutants can include single amino acid changes (point mutations) (e.g., replacement of one or more of the lysine's with a corresponding arginine), and the like. Mutants can be generated using standard techniques of molecular biology.

In an embodiment, chlorotoxin was labeled with ¹⁸F by coupling the chlorotoxin with N-succinimidyl-4-¹⁸F-fluorobenzoate (¹⁸F-SFB) (e.g., via the ε-amino group of lysine residue in Cltx) under slightly basic conditions (e.g., pH of about 8.5). Additional details regarding the ¹⁸F-labeled chlorotoxin are described in Example 1.

In addition, other ¹⁸F labeling strategies can be used, including, but not limited to, ¹⁸F labeling through an amine group, ¹⁸F labeling through a thiol group, and the like. In an embodiment, the ¹⁸F labeling strategy can include Boc-aminooxyacetic acid (AO), where AO can be coupled with the lead proteins during the solid phase peptide synthesis (SPPS). The resulting bioconjugates (AO-protein) will then be radiolabeled with 4-¹⁸F-FBA and generate 4-^{18/19}F-fluorobenzaldehyde conjugated aminooxy-chlorotoxin.

### Methods of Use

Embodiments of this disclosure include, but are not limited to: methods of imaging MMP-2 positive tissue; methods of imaging MMP-2 positive precancerous tissue, MMP-2 positive cancer, and MMP-2 positive tumors; methods of treating MMP-2 positive precancerous tissue, MMP-2 positive cancer, and MMP-2 positive tumors; methods of diagnosing MMP-2 positive precancerous tissue, MMP-2 positive cancer, and MMP-2 positive tumors; methods of monitoring the progress of MMP-2 positive precancerous tissue, MMP-2 positive cancer, and MMP-2 positive tumors; methods of imaging abnormal MMP-2 positive tissue and MMP-2 positive disease states; and the like. In addition, embodiments of the present disclosure include methods of detecting a signal from labeled chlorotoxin.

Embodiments of the present disclosure can be used to detect, study, monitor, evaluate, and/or screen, biological events in vivo or in vitro, such as, but not limited to, diseases involved in the expression of MMP-2 (MMP-2 positive precancerous tissue, cancer, tumors, and disease) and related biological events and labeled chlorotoxin. It should be noted that labeled chlorotoxin is referred to as “¹⁸F-labeled chlorotoxin” to illustrate embodiments of the present disclosure. It should be noted that other labels could be used to label chlorotoxin and perform in a similar manner as ¹⁸F-labeled chlorotoxin. In addition, if another label is used the imaging technique (e.g., SPECT, MRI, or the like) may change as well (e.g., if the label is a SPECT label, the imaging technique would be SPECT).

In general, the ¹⁸F-labeled chlorotoxin can be used in imaging cancer cells or tissue. For example, the ¹⁸F-labeled chlorotoxin is provided to a host in an amount effective to result in uptake of the compound into the cells or tissue of interest (e.g., MMP-2 positive tissue, cancer, and tumors). The host is then exposed to an appropriate PET source (e.g., a light source) after a certain amount of time. The cells or tissue that take up the ¹⁸F-labeled chlorotoxin can be images by detecting the signal from the ¹⁸F-labeled chlorotoxin using a PET imaging system.

In an embodiment, the ¹⁸F-labeled chlorotoxin can be used in imaging cancerous cells, precancerous cells, and tumors. It should be noted that the 18F-labeled chlorotoxin is preferentially taken up by MMP-2 cancerous cells, precancerous cells, and tumors. Thus, the ¹⁸F-labeled chlorotoxin may find use both in diagnosing cancer and/or in treating cancer.

In diagnosing the presence of cancerous cells, precancerous cells, and tumors in a subject, ¹⁸F-labeled chlorotoxin is administered to the subject in an amount effective to result in uptake of the ¹⁸F-labeled chlorotoxin into the cells so that a detectable signal could be produced. After administration of the ¹⁸F-labeled chlorotoxin, the cells that take up the ¹⁸F-labeled chlorotoxin can be imaged by detecting the ¹⁸F-labeled chlorotoxin using PET imaging. Embodiments of the present disclosure can non-invasively image tissue throughout an animal or patient.

In another embodiment, the ¹⁸F-labeled chlorotoxin can be used in treating cancer that has been previously diagnosed by a method described herein or by another method. The ¹⁸F-labeled chlorotoxin finds use in both surgical treatment and in chemical treatment of cancerous tissue. In patients where cancerous tissue is to be surgically removed, the ¹⁸F-labeled chlorotoxin is administered prior to and/or coincident with the surgical procedure. The cancerous tissue is appropriately irradiated and an attending medical provider can then directly visualize the illuminated tissue.

The ¹⁸F-labeled chlorotoxin can also find use in patients undergoing chemotherapy, to aid in visualizing the response of tumor tissue to the treatment. In this embodiment, the cancer tissue is typically visualized and sized prior to treatment, and periodically during chemotherapy to monitor the tumor size.

The ¹⁸F-labeled chlorotoxin also finds use as a screening tool in vitro to select compounds for use in treating cancer. The size of an in vitro tumor can be easily monitored in the presence of candidate drugs by incubating the cells with the ¹⁸F-labeled chlorotoxin during or after incubation with one or more candidate drugs.

It should be noted that the amount effective to result in uptake of the compound into the cells or tissue of interest will depend upon a variety of factors, including for example, the age, body weight, general health, sex, and diet of the host; the time of administration; the route of administration; the rate of excretion of the specific compound employed; the duration of the treatment; the existence of other drugs used in combination or coincidental with the specific composition employed; and like factors well known in the medical arts.

### Kits

This disclosure encompasses kits that include, but are not limited to, labeled (e.g., radiolabeled) chlorotoxin and directions (written instructions for their use). The components listed above can be tailored to the particular biological event (e.g., MMP-2 positive tissue, cancers, and diseases) to be monitored as described herein. The kit can further include appropriate buffers and reagents known in the art for administering various combinations of the components listed above to the host cell or host organism.

### Dosage Forms

Unit dosage forms of the pharmaceutical compositions of this disclosure may be suitable for oral, mucosal (e.g., nasal, sublingual, vaginal, buccal, or rectal), parenteral (e.g., intramuscular, subcutaneous, intravenous, intra-arterial, or bolus injection), topical, or transdermal administration to a patient. Examples of dosage forms include, but are not limited to: tablets; caplets; capsules, such as hard gelatin capsules and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the compositions of the disclosure typically vary depending on their use. For example, a parenteral dosage form may contain smaller amounts of the active ingredient than an oral dosage form used to treat the same condition or disorder. These and other ways in which specific dosage forms encompassed by this disclosure vary from one another will be readily apparent to those skilled in the art (See, e.g., Remington's Pharmaceutical Sciences, 18th ed., Mack Publishing, Easton, Pa. (1990)).

Typical compositions and dosage forms of the compositions of the disclosure can include one or more excipients. Suitable excipients are well known to those skilled in the art of pharmacy or pharmaceutics, and non-limiting examples of suitable excipients are provided herein. Whether a particular excipient is suitable for incorporation into a composition or dosage form depends on a variety of factors well known in the art including, but not limited to, the way in which the dosage form will be administered to a patient. For example, oral dosage forms, such as tablets or capsules, may contain excipients not suited for use in parenteral dosage forms. The suitability of a particular excipient may also depend on the specific active ingredients in the dosage form. For example, the decomposition of some active ingredients can be accelerated by some excipients, such as lactose, or by exposure to water. Active ingredients that include primary or secondary amines are particularly susceptible to such accelerated decomposition.

The disclosure encompasses compositions and dosage forms of the compositions of the disclosure that can include one or more compounds that reduce the rate by which an active ingredient will decompose. Such compounds, which are referred to herein as “stabilizers,” include, but are not limited to, antioxidants such as ascorbic acid, pH buffers, or salt buffers. In addition, pharmaceutical compositions or dosage forms of the disclosure may contain one or more solubility modulators, such as sodium chloride, sodium sulfate, sodium or potassium phosphate, or organic acids. An exemplary solubility modulator is tartaric acid.

Like the amounts and types of excipients, the amounts and specific type of active ingredient in a dosage form may differ depending on various factors. It will be understood, however, that the total daily usage of the compositions of the present disclosure will be decided by the attending physician or other attending professional within the scope of sound medical judgment. The specific effective dose level for any particular host will depend upon a variety of factors, including for example, the activity of the specific composition employed; the specific composition employed; the age, body weight, general health, sex, and diet of the host; the time of administration; the route of administration; the rate of excretion of the specific compound employed; the duration of the treatment; the existence of other drugs used in combination or coincidental with the specific composition employed; and like factors well known in the medical arts. For example, it is well within the skill of the art to start doses of the composition at levels lower than those required to achieve the desired effect and to gradually increase the dosage until the desired effect is achieved.

### EXAMPLE

Now having described the embodiments of the disclosure, in general, the following example describes some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Experimental

No-carrier-added [¹⁸F]Fluoride was produced at the Molecular Imaging Program at Stanford, PETtrace cyclotron (General Electric Health Care, Waukesha, Wis.) by irradiation of enriched [¹⁸O]water via the ¹⁸O(p,n)¹⁸F nuclear reaction. Cltx peptide was purchased from AnaSpec (San Jose, Calif.). All other reagents were purchased from Sigma-Aldrich Chemical Co. Matrix-assisted laser desorption/ionization time of flight mass spectrometry (MALDI-TOF-MS) was performed on a Perseptive Voyager-DE RP Biospectrometry instrument (Framingham, Mass.) by Stanford Protein and Nucleic Acid Biotechnology Facility. HPLC was performed on a Dionex Summit® HPLC system (Dionex Corporation, Sunnyvale, Calif.) equipped with a 170U 4-Channel UV-Vis absorbance detector and radioactive detector (Carroll & Ramsey Associates, model 105S, Berkeley, Calif.). UV detection wavelengths were 225 nm and 280 nm for all the experiments. Both semi-preparative (GRACE Vydac C18, 9.4 mm×250 mm, CAT# 218TP510) and analytical (Dionex Acclaim® 120 C18, 4.6 mm×250 mm) RP-HPLC columns were used. The mobile phase was solvent A, water/0.1% trifluoroacetic acid (TFA), and solvent B, acetonitrile/0.1% TFA. A CRC-15R PET dose calibrator (Capintec Inc., Ramsey, N.J.) was used for all radioactivity measurements.

### Cell Lines and Tumor Xenografts

MMP-2 positive tumor cell lines, including U87MG human glioblastoma cells, C6 rat glioma cells, B16F10 murine melanoma cells, and MDA-MB-435 human breast cancer cells were all obtained from American Type Culture Collection (Manassas, Va.). U87MG, C6, and B16F10 cells were cultured in Dulbecco's modified Eagle high glucose medium supplemented with 10% fetal bovine serum (FBS) and 1% penicillin-streptomycin (DMEM, Invitrogen Life Technologies, Carlsbad, Calif.). MDA-MB-435 was cultured in Leibovitz's L-15 medium with 2 mM L-glutamine supplemented with 0.01 mg/mL insulin, 10% FBS, and 1% penicillin-streptomycin (Invitrogen Life Technologies, Carlsbad, Calif.). All the cell lines were maintained in a humidified atmosphere of 5% CO₂ at 37° C., with the medium changed every other day. A confluent monolayer was detached with trypsin and dissociated into a single cell suspension for further cell culture.

Female athymic nude mice (nu/nu), obtained from Charles River Laboratories, Inc. (Cambridge, Mass.) at 4-6 weeks of age, were subcutaneously injected in the right or left shoulder with 5×10⁶ U87MG glioblastoma cells, or B16F10, or C6, or MDA-MB-435 cells suspended in 100 μL of phosphate buffered saline (PBS, 0.01 mol/L; pH, 7.4). When the tumors reached 0.4-0.6 cm in diameter, the tumor bearing mice were subjected to in vivo imaging studies.

Radiosynthesis and HPLC purification of ¹⁸F-FB-Cltx

The radiosynthesis of ¹⁸F-fluorobenzoate conjugated chlorotoxin (¹⁸F-FB-Cltx) is shown in FIG. 2. N-succinimidyl-4-¹⁸F-fluorobenzoate (¹⁸F-SFB) was first synthesized by using the procedure reported before (Chen X, Tohme M, Park R, Hou Y, Bading J R, Conti P S. Micro-PET imaging of alphavbeta3-integrin expression with 18F-labeled dimeric RGD peptide. Mol. Imaging. 2004; 3:96-104 incorporated herein by reference). The HPLC purified ¹⁸F-SFB in acetonitrile (500 μL) was then added to the Cltx (50 μg in 25 μL H₂O) and sodium borate buffer (0.1 M. pH=8.5, 500 μL). After being incubated at 40° C. for 60 min, the reaction was stopped by adding 50 μL trifluoroacetic acid (TFA). The solution was injected onto a semipreparative HPLC column (the flow rate was 5 ml/min, with the mobile phase starting from 10% solvent B (CH₃CN/0.1% TFA) and 90% solvent A (H₂O/0.1% TFA) (0-3 min) to 40% solvent B and 60% solvent A at 33 min, then going to 85% solvent B and 15% solvent A (33-36 min), maintaining this solvent composition for another 3 min (36-39 min), and returning to initial solvent composition by 42 min). Pure ¹⁸F-FB-Cltx, eluted out the column with a retention time of 18.7 min, was collected in a small round bottle and dried in a rotary evaporator. The product was finally reconstituted in phosphate-buffered saline (PBS) and passed through a 0.22-mm Millipore filter into a sterile multidose vial for further studies.

### Animal Biodistribution Studies

For biodistribution studies, the nude mice bearing B16F10 mouse melanoma and MDA-MB-435 breast cancer (n=3 for each group) were injected with about 20 μCi of ¹⁸F-FB-Cltx through the tail vein and sacrificed at 3.5 h post injection. Tumor and normal tissues of interest were removed and weighed, and their radioactivity was measured in a gamma-counter. The radioactivity uptake in the tumor and normal tissues was expressed as a percentage of the injected radioactive dose per gram of tissue (% ID/g).

### MicroPET Imaging

PET imaging of normal nude mice was performed on a microPET R4 rodent model scanner (Concorde Microsystems Inc, Knoxville, Tenn.). The mice were injected with about 50 μCi of ¹⁸F-FB-Cltx via the tail vein. At 30 min, 1 hr, 2 hr and 3 hr post injection, the mice were anesthetized with 2% isoflurane, and placed in the prone position and near the center of the filed of view of microPET. The 10-min static scans were obtained and the images were reconstructed by a two-dimensional ordered subsets expectation maximum (OSEM) algorithm. Regions of interest (ROIs) were then drawn over the tumor on decay-corrected whole-body coronal images. The counts per pixel per minute were obtained from the ROI and converted to counts per milliliter per minute by using a calibration constant. By assuming a tissue density of 1 g/mL, the ROIs were converted to counts/g/min. An image ROI-derived percentage ID per gram of tissue (% ID/g) was then determined by dividing counts per gram per minute with injected dose (ID).

### Statistical Method

Statistical analysis was performed using the Student's t-test for unpaired data. A 95% confidence level was chosen to determine the significance between groups, with P<0.05 being significantly different.

### Results and Discussion

### Radiochemistry

The synthesis of ¹⁸F-FB-Cltx conjugate (FIG. 2) was achieved through coupling of ¹⁸F-SFB with the ε-amino group of the lysine residue in the Cltx. The desired product was purified by semi-preparative HPLC, and the purity of target compound was generally obtained in 20% yield and over 95% purity. The retention time of ¹⁸F-FB-Cltx was found to be 18.7 minutes, which was consistent with the non-radioactive FB-Cltx (the measured molecular weight (MW) m/z=4119.2 for [M+H]⁺, expected MW: 4119.0). The specific radioactivity of ¹⁸F-FB-Cltx was estimated by radio-HPLC to be 200-250 TBq/mmol.

### Biodistribution Studies

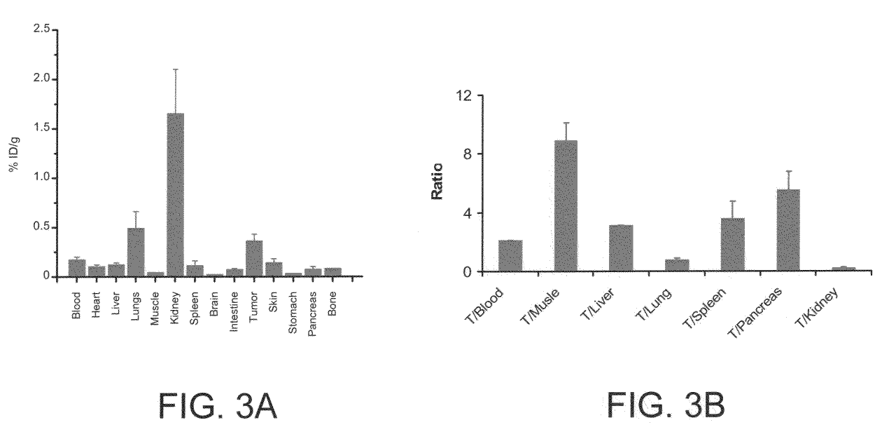

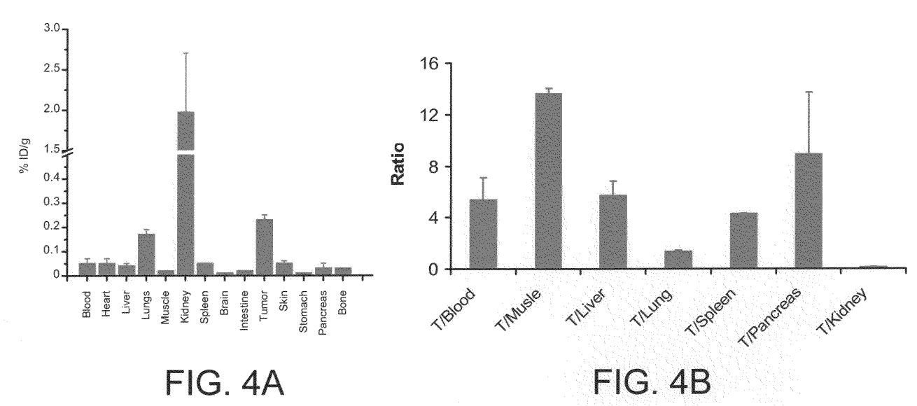

The in vivo biodistribution of ¹⁸F-FB-Cltx was examined in either a MDA-MB-435 breast cancer or B16F10 melanoma-bearing mouse model. Biodistribution of the radiolabeled Cltx at 3.5 h was obtained in MDA-MB0435 and B16F10 tumor models and are shown in FIGS. 3A and 4A, respectively. The tumor to normal organ ratios of ¹⁸F-FB-Cltx in these two tumor models are also shown in FIGS. 3B and 4B.

In both tumor models, the majority of ¹⁸F-FB-Cltx was cleared from mouse at 3.5 h. The kidney is the organ that shows the highest uptake (1.65±0.45 and 1.97±0.73% ID/g for MDA-MB-435 and B16F10, respectively). Low liver activity (0.12±0.02 for MDA-MB-435 and 0.05±0.01 for B16F10) was also observed. The radiofluorinated Cltx also shows moderate tumor uptake (0.36±0.07 for MDA-MB-435 and 0.23±0.02 for B16F10) and low blood and muscle uptake (less than 0.05 ID/g), resulting in reasonable tumor-to-blood and tumor-muscle ratios (FIGS. 3B and 4B).

### MicroPET Imaging of ¹⁸F-FB-Cltx

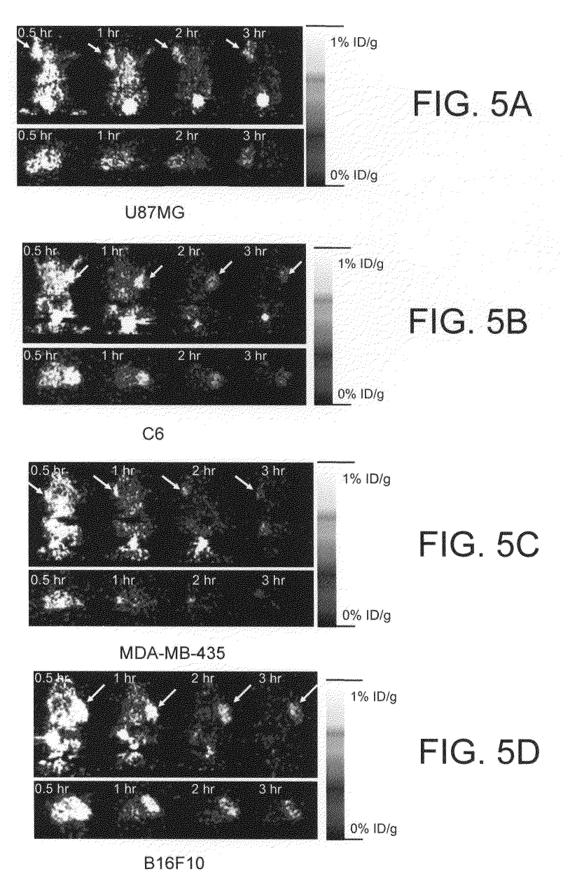

FIG. 5 shows coronal and transverse microPET images of normal nude mice bearing U87MG (A), C6 (B), MDA-MB-435 (C), or B16F10 (D) tumors at different times post injection of 50 μCi of the ¹⁸F-FB-Cltx. All micro-PET images were decay corrected. The ¹⁸F-FB-Cltx clearly localized in these tumor models. Moreover, high activity was also observed to localize in kidneys and bladder, indicating that the tracer was mainly cleared out through urine system.

### CONCLUSION

In conclusion, ¹⁸F-FB-Cltx was synthesized in high radiochemical purity. MicroPET imaging studies demonstrated that the probe has great potential for imaging MMP2 expression in disease.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

### Sequence Listing:

SEQ ID No: 1:
Met-Cys-Met-Pro-Cys-Phe-Thr-Thr-Asp-His-Gln-Met-
Ala-Arg-Lys-Cys-Asp-Asp-Cys-Cys-Gly-Gly-Lys-Gly-
Arg-Gly-Lys-Cys-Tyr-Gly-Pro-Gln-Cys-Leu-Cys-Arg

We claim the following:

1. A method for imaging MMP-2 positive tissue that includes:
contacting a MMP-2 positive tissue with a labeled chlorotoxin; and
imaging the tissue with an imaging system.

2. The method of claim 1, wherein the labeled chlorotoxin is a radio-labeled chlorotoxin, wherein the radio-label is selected from: ¹⁸F, ¹²⁴I, ¹²³I, ¹²⁵I, ¹³¹I, ^{76/77}Br, ⁶⁴Cu, ⁸⁶Y, ⁸⁹Zr, ⁶⁸Ga, ⁹⁹Tc, ¹¹¹In, ^{186/188}Re, ¹⁷⁷Lu, ¹⁵³Sm, or 90Y.

3. The method of claim 1, wherein the labeled chlorotoxin is a ¹⁸F-labeled chlorotoxin, analogs thereof, portions thereof, mutants thereof, or varients thereof.

4. The method of claim 1, wherein the labeled chlorotoxin is a ¹⁸F-labeled chlorotoxin.

5. The method of claim 4, wherein the labeled chlorotoxin is labeled with ¹⁸F by coupling the chlorotoxin with N-succinimidyl-4-¹⁸F-fluorobenzoate (¹⁸F-SFB), wherein chlorotoxin has an amino acid sequence SEQ ID NO: 1.

6. The method of claim 5, wherein the imaging system is selected from a PET imaging system or a SPECT imaging system.

7. The method of claim 6, wherein the imaging can be performed in vivo or in vitro.

8. The method of claim 8, wherein the MMP-2 tissue is selected from: MMP-2 positive precancerous cells, MMP-2 positive cancer tissue, or MMP-2 positive tumor tissue.

9. A method of diagnosing the presence of one or more of MMP-2 positive precancerous cells, MMP-2 positive cancerous cells, MMP-2 positive tumor cells, and MMP-2 positive diseases in a tissue comprising:
contacting a tissue with a labeled chlorotoxin; and
imaging the tissue with an imaging system.

10. The method of claim 9, wherein the labeled chlorotoxin is a ¹⁸F-labeled chlorotoxin, analogs thereof, portions thereof, mutants thereof, or varients thereof.

11. The method of claim 9, wherein the labeled chlorotoxin is a ¹⁸F-labeled chlorotoxin.

12. The method of claim 11, wherein the labeled chlorotoxin is labeled with ¹⁸F by coupling the chlorotoxin with N-succinimidyl-4-¹⁸F-fluorobenzoate (¹⁸F-SFB), wherein chlorotoxin has an amino acid sequence SEQ ID NO: 1.

13. A method of monitoring the progress of one or more of MMP-2 positive precancerous cells, MMP-2 positive cancerous cells, MMP-2 positive tumor cells, and MMP-2 positive diseases in a tissue comprising:
contacting a MMP-2 positive tissue with a labeled chlorotoxin; and
imaging the MMP-2 positive tissue with an imaging system.

14. A pharmaceutical composition for imaging one or more of MMP-2 positive precancerous cells, MMP-2 positive cancerous cells, MMP-2 positive tumor cells, and MMP-2 positive diseases, comprising: a labeled chlorotoxin.

15. The pharmaceutical composition of claim 14, wherein the labeled chlorotoxin is a radio-labeled chlorotoxin, wherein the radio-label is selected from: ¹⁸F, ¹²⁴I, ¹²³I, ¹²⁵I, ¹³¹I, ^{76/77}Br, ⁶⁴Cu, ⁸⁶Y, ⁸⁹Zr, ⁶⁸Ga, ⁹⁹Tc, ¹¹¹In, ^{186/188}Re, ¹⁷⁷Lu, ¹⁵³Sm, or 90Y.

16. The pharmaceutical composition of claim 14, wherein the labeled chlorotoxin is a ¹⁸F-labeled chlorotoxin, analogs thereof, portions thereof, mutants thereof, or varients thereof.

17. The pharmaceutical composition of claim 17, wherein the labeled chlorotoxin is a ¹⁸F-labeled chlorotoxin.

18. The pharmaceutical composition of claim 17, wherein the labeled chlorotoxin is labeled with ¹⁸F by coupling the chlorotoxin with N-succinimidyl-4-¹⁸F-fluorobenzoate (¹⁸F-SFB), wherein chlorotoxin has an amino acid sequence SEQ ID NO: 1.

19. A composition, comprising: a labeled chlorotoxin, analogs thereof, portions thereof, mutants thereof, or varients thereof.

20. The composition of claim 19, wherein the labeled chlorotoxin is a radio-labeled chlorotoxin, wherein the radio-label is selected from: ¹⁸F, ¹²⁴I, ¹²³I, ¹²⁵I, ¹³¹I, ^{76/77}Br, ⁶⁴CU, ⁸⁶Y, ⁸⁹Zr, ⁶⁸Ga, ⁹⁹Tc, ¹¹¹In, ^{186/188}Re, ¹⁷⁷Lu, ¹⁵³Sm, or 90Y.

21. The composition of claim 19, wherein the labeled chlorotoxin is a ¹⁸F-labeled chlorotoxin, analogs thereof, portions thereof, mutants thereof, or varients thereof.

22. The composition of claim 21, wherein the labeled chlorotoxin is a ¹⁸F-labeled chlorotoxin.

23. The composition of claim 22, wherein the labeled chlorotoxin is labeled with ¹⁸F by coupling the chlorotoxin with N-succinimidyl-4-¹⁸F-fluorobenzoate (¹⁸F-SFB), wherein chlorotoxin has an amino acid sequence SEQ ID NO: 1.

24. A kit for imaging MMP-2 positive precancerous cells, cancer, tumor tissue and MMP-2 positive diseases, comprising: a ¹⁸F-labeled chlorotoxin and directions for use.

25. The composition of claim 24, wherein the labeled chlorotoxin is labeled with ¹⁸F by coupling the chlorotoxin with N-succinimidyl-4-¹⁸F-fluorobenzoate (¹⁸F-SFB), wherein chlorotoxin has an amino acid sequence SEQ ID NO: 1.


SEQUENCE LISTING (1 sequence)

SEQ ID NO: 1 - protein, 36 aa, Scorpion Venom
MCMPCFTTDHQMARKCDDCCGGKGRGKCYGPQCLCR